(19) United States
(12) Patent Application Publication
(10) Pub. No.: US 2009/0286750 A1
(43) Pub. Date: Nov. 19, 2009

(54) USE OF RIBOSE IN LESSENING THE CLINICAL SYMPTOMS OF ABERRANT FIRING OF NEURONS

(76) Inventors: Robert V. Kasubick, Red Oak, IA (US); John A. St. Cyr, Coon Rapids, MN (US)

Correspondence Address: MUETING, RAASCH & GEBHARDT, P.A., P.O. BOX 581336, MINNEAPOLIS, MN, 55458-1336, US

(21) Appl. No.: 12/466,893

(22) Filed: May 15, 2009

Related U.S. Application Data
(60) Provisional application No. 61/127,843, filed on May 16, 2008.

(51) Int. Cl. A61K 31/7004

(52) U.S. Cl. 514/23

(57) ABSTRACT

A method for treating a mammal suffering from a condition caused by aberrant neuron firing is disclosed herein. The method includes administering (e.g., orally administering) to the mammal (e.g., a human patient) in need thereof a therapeutically effective amount of a composition including D-ribose.

This application claims the benefit of U.S. Provisional Application Ser. No. 61/127,843, filed May 16, 2008, which is herein incorporated by reference in its entirety.

### BACKGROUND

The neurons of the central nervous system (CNS) which includes the brain and the spinal cord, are sequestered against injurious chemicals by the blood brain barrier (BBB), a term used for the barrier between peripheral circulation and the neurons of the CNS. The barrier is formed by tight junctions within the brain capillary endothelial membranes and surveillances the passage of molecules, including even small molecules, and accepting those that have specific receptors which facilitate the transport of the specific molecule across the BBB. An example of the latter is the transportation of the insulin molecule, which is not true for other compounds, although lipids may cross the BBB more readily than other kinds of molecules. For example, no definitive transport receptor or mechanisms for carbohydrates in general is known; however, some carbohydrates can cross this barrier for metabolic uses, such as the molecule glucose which passes most likely via the Glu-4 transportation mechanism. It is possible for molecules or compounds that would otherwise be excluded from entrance across the BBB and influence neurons of the CNS to be “piggybacked” passively by mass action and therefore cross this guarded barrier with an acceptable molecule or compound.

Neurons in the CNS are sometimes found to fire aberrantly, that is, without uniformity or volition to a reflex response to a stimulus. Such conditions can be found in diseases such as cerebral palsy, where injury to the brain can occur during gestation, during the birthing interval, hypoxia at any age or even with aging, which can result in uncontrolled, aberrant, muscular contractions. This arena presents a spectrum of aberrant neuron firing, such as in human patients with cerebral palsy, mammals (e.g., human patients, horses, dogs) with epilepsy, human patients with states of neuronal chemical alterations/imbalances, human patients with cerebral neural toxicity events, human patients with congenital malformations, and human patients with Parkinson's disease. In the lesser extreme, human patients with Restless Legs Syndrome (RLS), also referred to as nocturnal myoclonus, do experience states of CNS motor dysfunction, which can be useful as a model for studying these more severe conditions. RLS is clinically manifested by unpleasant sensations and pain, accompanied by an uncontrollable urge for single or repetitive movements for relief.

In general, these neuronal/neuromuscular conditional states are debilitating to a greater or lesser degree. Therapeutic options to lessen the manifested symptoms can produce varying degrees of success. Many patients afflicted with one of these debilitating neural states search for treatments to aid in relieving their discomfort with hopes to lessening the daily experiences of motor dysfunctional chharacteristics. Because of this unanswered need, patients have explored the use of alternative solutions, including nutritional supplements, in the hope of even partially lessening existing symptoms, and improve their daily quality of life measures. However, safe, easy to administer, and readily available nutritional supplements with sound evidence for efficacy have heretofore not been identified.

### SUMMARY

In one embodiment, the present disclosure provides a method for treating a human patient having restless legs syndrome. The method includes administering to the patient in need thereof a therapeutically effective amount of a composition including D-ribose. For example, in embodiments including human patients suffering from symptoms of restless legs syndrome, two 5 gram doses of D-Ribose, taken morning and noon, followed by a dose of eight grams of D-Ribose can be sufficient to lesson the symptoms of RLS.

In another embodiment, the present disclosure provides a method for treating a human patient having cerebral palsy. The method includes administering to the patient in need thereof a therapeutically effective amount of a composition including D-ribose. For example, in embodiments including patients suffering from cerebral palsy, more frequent pulse 5 gram doses of D-Ribose may be indicated. The exact frequency for dosage may be determined empirically for each patient.

In still another embodiment, the present disclosure provides a method for treating a mammal having epilepsy. The method including administering to the mammal in need thereof a therapeutically effective amount of a composition including D-ribose. For example, in embodiments including mammals (e.g., human patients) suffering from epilepsy, more frequent pulse 5 gram doses of D-Ribose may be indicated. The exact frequency for dosage may be determined empirically for each mammal, including humans.

In yet another embodiment, the present disclosure provides a method for treating a mammal suffering from a condition caused by aberrant neuron firing. The method including administering to the mammal in need thereof a therapeutically effective amount of a composition including D-ribose. D-ribose has been found to relieve the symptoms of aberrant motor neuron firing. An effective amount of D-ribose can be administered to mammals (e.g., human patients, horses, dogs) suffering from the symptoms of aberrant motor neuron firing. The effective required dose should at least maintain a serum D-ribose level 4 to 40 mg/dl for a long enough time such that the residual beneficial effect lasts until the next dose. For embodiments including oral administration to human patients, the recommended 5 gram dose of D-ribose taken three to ten times daily can lessen the occurrence and severity of the symptoms with an improvement in quality of life. For overnight night relief, the last dose of the day can be increased to 8 to 10 grams of D-ribose, and optionally taken with a source of glucose, such as fruit juice.

D-Ribose may be administered in the form of powders, lozenges, capsules or may be incorporated in a liquid drink. Time-release tablets or capsules may maintain the serum levels to the required level when taken fewer times a day.

The infusion of D-ribose can also involve the use of an infusible pump apparatus. A pump device can deliver drugs, nutrients, or biologics, even at low doses at a constant or controllable rate; thereby maintaining a relatively constant serum level of the infusible compound or molecule without interruption. An infusion catheter is commonly threaded into a central vein with the other end either exiting from the skin to an external pump, which the pump can be attached to the person for ease of mobility or to a stationary pump. The concept of an implantable pump placed subcutaneously is actively being pursued, which would require its reservoir to be filled from time to time, sterilely through the skin.

Infusible pumps are spring-operated or elastomeric. Spring-operated pumps use mechanical positive-pressure and are commonly used for intermittent antibiotic therapy, chemotherapy and analgesic therapies. The use of elastomeric pump for continuous delivery of a solution is growing in popularity. These pumps, commonly used for drug therapies, are lightweight, easy to use, easily portable, and use a positive pressure system of delivering a fluid at a constant rate for accuracy with the maintenance of a chosen serum level of the infusible solution. One of the most common uses of delivery for medication is a pump to deliver insulin; another is for the delivery of chemotherapeutic agents; and yet another is used for delivery of pain medication. Such pumps would be especially useful for delivering pyrogen-free D-Ribose to those patients who may require continuously high serum levels of D-Ribose for the control of symptoms.

As used herein, “a,” “an,” “the,” and “at least one” are used interchangeably and mean one or more than one.

As used herein, the term “comprising,” which is synonymous with “including” or “containing,” is inclusive, open-ended, and does not exclude additional unrecited elements or method steps.

### DETAILED DESCRIPTION OF ILLUSTRATIVE EMBODIMENTS

In one embodiment, the present disclosure provides a method for treating a human patient having restless legs syndrome. The method includes administering (e.g.,orally) to the patient in need thereof a therapeutically effective amount of a composition including D-ribose. It should be understood that the composition including D-ribose is not a cure for restless legs syndrome, but is expected to at least partially ameliorate symptoms at therapeutically effective doses. Thus, for embodiments in which the human patient has restless legs syndrome, a therapeutically effective amount of the composition means an amount which, when administered to the patient, results in the patient experiencing at least partial relief from the symptoms. In preferred embodiments, the patient can experience substantial or even total relief from the symptoms.

For embodiments in which the human patient has restless legs syndrome, the composition including D-ribose can be administered for at least one week, at least 3 weeks, or longer (e.g., chronic administration). In certain embodiments, the therapeutically effective amount of the composition includes 4 to 50 grams of D-ribose per day. In some embodiments, the therapeutically effective amount of the composition is administered in 1 to 10 doses per day, in certain embodiments in 1 to 4 doses per day, and in other certain embodiments three times a day. The D-ribose content of each dose of the composition can be calculated from the desired total daily dose of D-ribose and the number of desired doses per day, and can be any convenient size (e.g., 1 gram, 1.5 grams, 2 grams, 5 grams, 6 grams of D-ribose per dose, or even higher). In some embodiments, administering the therapeutically effective amount of the composition includes administering five grams of D-ribose three times a day. In certain embodiments, administering the therapeutically effective amount of the composition consists essentially of administering five grams of D-ribose three times a day (e.g., administering a composition that can include inert and other ingredients that are not substantially active towards treating RLS). In other certain embodiments, administering the therapeutically effective amount of the composition consists of administering five grams of D-ribose three times a day (e.g., no other ingredients administered). In some embodiments the administered composition does not include folic acid and/or folate. In certain embodiments, the administered composition does not include any of vitamin B6, vitiamin B12, folic acid, or folate. In other certain embodiments, the administered composition does not include a B group vitamin. In other certain embodiment, the administered composition does not include one or more of proteins, fatty acids, or lipids.

In one embodiment, the present disclosure provides a method for treating a human patient having cerebral palsy. The method includes administering (e.g.,orally) to the patient in need thereof a therapeutically effective amount of a composition including D-ribose. It should be understood that the composition including D-ribose is not a cure for cerebral palsy, but is expected to at least partially ameliorate symptoms at therapeutically effective doses. Thus, for embodiments in which the human patient has cerebral palsy, a therapeutically effective amount of the composition means an amount which, when administered to the patient, results in the patient experiencing at least partial relief from the symptoms. In preferred embodiments, the patient can experience substantial or even total relief from the symptoms.

For embodiments in which the human patient has cerebral palsy, the composition including D-ribose can be administered for at least one week, at least 3 weeks, or longer (e.g., chronic administration). In some embodiments, the therapeutically effective amount of the composition includes 4 to 50 grams of D-ribose per day, and in certain embodiments 15 to 20 grams of D-ribose per day. In some embodiments, the therapeutically effective amount of the composition is administered in 3 to 10 doses per day, in certain embodiments in 3 to 8 doses per day, and in other certain embodiments three times a day. The D-ribose content of each dose of the composition can be calculated from the desired total daily dose of D-ribose and the number of desired doses per day, and can be any convenient size (e.g., 1 gram, 1.5 grams, 2 grams, 5 grams, 6 grams of D-ribose per dose, or even higher). In some embodiments, administering the therapeutically effective amount of the composition includes administering five grams of D-ribose three times a day. In certain embodiments, administering the therapeutically effective amount of the composition consists essentially of administering five grams of D-ribose three times a day (e.g., administering a composition that can include inert and other ingredients that are not substantially active towards treating cerebral palsy). In other certain embodiments, administering the therapeutically effective amount of the composition consists of administering five grams of D-ribose three times a day (e.g., no other ingredients administered). In some embodiments the administered composition does not include folic acid and/or folate. In certain embodiments, the administered composition does not include any of vitamin B6, vitiamin B12, folic acid, or folate. In other certain embodiments, the administered composition does not include a B group vitamin. In other certain embodiment, the administered composition does not include one or more of proteins, fatty acids, or lipids.

In one embodiment, the present disclosure provides a method for treating a mammal having epilepsy. The method includes administering (e.g.,orally) to the mammal in need thereof a therapeutically effective amount of a composition including D-ribose. It should be understood that the composition including D-ribose is not a cure for epilepsy, but is expected to at least partially ameliorate symptoms at therapeutically effective doses. Thus, for embodiments in which the mammal has epilepsy, a therapeutically effective amount of the composition means an amount which, when administered to the mammal, results in the mammal experiencing at least partial relief from the symptoms. In preferred embodiments, the mammal can experience substantial or even total relief from the symptoms.

For embodiments in which the mammal is a human patient having epilepsy, the composition including D-ribose can be administered for at least one week, at least 3 weeks, or longer (e.g., chronic administration). In some embodiments, the therapeutically effective amount of the composition includes 4 to 50 grams of D-ribose per day, and in certain embodiments 15 to 20 grams of D-ribose per day. In some embodiments, the therapeutically effective amount of the composition is administered in 3 to 10 doses per day, in certain embodiments in 3 to 8 doses per day, and in other certain embodiments three times a day. The D-ribose content of each dose of the composition can be calculated from the desired total daily dose of D-ribose and the number of desired doses per day, and can be any convenient size (e.g., 1 gram, 1.5 grams, 2 grams, 5 grams, 6 grams of D-ribose per dose, or even higher). In some embodiments, administering the therapeutically effective amount of the composition includes administering five grams of D-ribose three times a day. In certain embodiments, administering the therapeutically effective amount of the composition consists essentially of administering five grams of D-ribose three times a day (e.g., administering a composition that can include inert and other ingredients that are not substantially active towards treating epilepsy). In other certain embodiments, administering the therapeutically effective amount of the composition consists of administering five grams of D-ribose three times a day (e.g., no other ingredients administered). In some embodiments the administered composition does not include folic acid and/or folate. In certain embodiments, the administered composition does not include any of vitamin B6, vitiamin B12, folic acid, or folate. In other certain embodiments, the administered composition does not include a B group vitamin. In other certain embodiment, the administered composition does not include one or more of proteins, fatty acids, or lipids. Dosage regimens for other mammals (e.g., horses, dogs) can be calculated based on known factors including body weight.

In one embodiment, the present disclosure provides a method for treating a mammal suffering from a condition caused by aberrant neuron firing. The method includes administering (e.g.,orally) to the mammal in need thereof a therapeutically effective amount of a composition including D-ribose. It should be understood that the composition including D-ribose is not a cure for the condition caused by aberrant neuron firing, but is expected to at least partially ameliorate symptoms at therapeutically effective doses. Thus, for embodiments in which the mammal has a condition caused by aberrant neuron firing, a therapeutically effective amount of the composition means an amount which, when administered to the mammal, results in the mammal experiencing at least partial relief from the symptoms. In preferred embodiments, the mammal can experience substantial or even total relief from the symptoms.

For embodiments in which the mammal is a human patient suffering from a condition caused by aberrant neuron firing, the composition including D-ribose can be administered for at least one week, at least 3 weeks, or longer (e.g., chronic administration). In certain embodiments, the therapeutically effective amount of the composition includes 4 to 50 grams of D-ribose per day, and in certain embodiments 15 to 20 grams of D-ribose per day. In some embodiments, the therapeutically effective amount of the composition is administered in 1 to 10 doses per day, in certain embodiments in 3 to 10 doses per day, and in other certain embodiments three times a day. The D-ribose content of each dose of the composition can be calculated from the desired total daily dose of D-ribose and the number of desired doses per day, and can be any convenient size (e.g., 1 gram, 1.5 grams, 2 grams, 5 grams, 6 grams of D-ribose per dose, or even higher). In certain embodiments, D-ribose can be administered using a pulse dosing regimen (e.g., multiple, repetitive doses, optionally within a short time period). A pulse dosing regimen can be useful for treating a mammal suffering declining health from a condition caused by aberrant neuron firing. In certain embodiments, the pulse dosing regimen can be escalated for mammals not responding to the pulse dosing regimen. In some embodiments, administering the therapeutically effective amount of the composition includes administering five grams of D-ribose three times a day. In certain embodiments, administering the therapeutically effective amount of the composition consists essentially of administering five grams of D-ribose three times a day (e.g., administering a composition that can include inert and other ingredients that are not substantially active towards treating a condition that includes aberrant neuron firing). In other certain embodiments, administering the therapeutically effective amount of the composition consists of administering five grams of D-ribose three times a day (e.g., no other ingredients administered). In some embodiments the administered composition does not include folic acid and/or folate. In certain embodiments, the administered composition does not include any of vitamin B6, vitiamin B12, folic acid, or folate. In other certain embodiments, the administered composition does not include a B group vitamin. In other certain embodiment, the administered composition does not include one or more of proteins, fatty acids, or lipids. Dosage regimens for other mammals (e.g., horses, dogs) can be calculated based on known factors including body weight.

The following examples are provided for illustrative purposes and do not limit the scope of this invention, which is the use of D-Ribose to relieve symptoms of CNS aberrant neuron firing. In all of these neuronal-erratic misfiring conditions, there exists an abnormal neuronal stimulatory activity within the CNS, which may involve both the brain and spinal cord. For purposes of this application, this abnormal neuronal firing state could center on an “injury” which may be trauma, toxicity, hypoxia, congenital abnormalities, aging or the like. However, in the case of most conditions, the nature of the “injury” remains unknown.

CNS neurons are cells that have extremely tight cellular junctions with surveillant capillary endothelial membranes, termed the blood brain barrier (BBB). The exact site of injury due to the above described etiologies is not understood; the ultimate deficit manifested in these neurons and accompanying surrounding tissues, including supporting glial cells or the endothelial membranes or in the junctions thereof remains to be determined. These ultimate neuronal deficits are reflective in clinical symptomatology. Whatever the extent of injury, we here propose that the subsequent clinical neural sequelae may reflect a deficit in the absolute levels, turnover rate or utilization of cellular ATP.

The pentose sugar D-Ribose has been extensively studied in skeletal and cardiac muscle with known benefits. It is theorized that D-Ribose accelerates the resynthesis of ATP through the pentose phosphate salvage pathway. For example, it has been shown that D-Ribose enhances cardiac recovery, e.g., U.S. Patent Application Publication No. 2004/0087515 A1 (Butler et al.) and raises the power output of exercising humans, e.g., U.S. Pat. No. 6,159,942 (St. Cyr et al.). These known results confirm the theory that D-Ribose plays an important role in achieving a more ideal cellular ATP state. For adequate levels of ATP are necessary to maintain cellular integrity and function. While it is known that the brain, like muscle, is an organ with high energy demands, nothing is known about the role of D-Ribose in the brain or whether D-Ribose readily transgresses the BBB. In fact, D-Ribose has been used to improve energy levels in normal individuals and cardiac patients for some time, but has not been reported to have an effect in this group of patients who suffer from symptoms due to aberrant motor neuron firing.

D-Ribose is found at low levels in the diet and normal serum levels are approximately 2 mg/dl. It has been found that D-Ribose is rapidly cleared from the blood, either by renal excretion, metabolic utilization, or conversion to D-Glucose. When infused for short periods at the concentrations of 60 mg/kg and 100 mg/kg, D-Ribose levels in the serum rose, in each case, to approximately 25 mg/dl. These doses of D-Ribose, for a 60 kilogram man, are 3.6 and 5 grams, respectively. Doses for larger or smaller size humans can be calculated based on their mass, and are typically 1 to 10 grams. It has been determined that the half life of D-Ribose in the serum was 11 minutes and 16 minutes, respectively. At higher oral doses of a constant consumption of D-Ribose, four hours of 83.2 or 166.7 mg/kg per hour, can likewise increase the serum D-Ribose level to 29.25 mg/dl and 35 mg/dl, respectively. Intravenous administration of high doses of D-Ribose can elevate the serum levels to as high as 60 mg/dl, while a further increase in dose to 222.2 mg/kg can produce a serum level as high as 86 mg/dl. See, for example, Gross et al., Klinische Wochenschrift (1991) 69:31-36.

It has long been known that D-Ribose supplementation can reflect a decrease in measured serum glucose levels. Previous studies have substantiated this finding, including the above mentioned investigation, that confirmed this premise, and furthermore, this decline in serum glucose reaches an ultimate lower level at a 25% drop, regardless of the measured serum level of D-Ribose. Therefore, when doses of D-Ribose higher than 5 grams are taken, the patient is advised to co-ingest a source of glucose, such as a fruit drink or candy.

The following example is offered to further illustrate various specific embodiments and techniques of the present invention. It should be understood, however, that many variations and modifications understood by those of ordinary skill in the art may be made while remaining within the scope of the present invention. Therefore, the scope of the invention is not intended to be limited by the following example.

### EXAMPLE 1

### Restless Legs Syndrome

Restless Legs Syndrome (RLS), also referred to as Nocturnal Myoclonus, is a comparatively mild example of a CNS/muscular alteration caused by an aberrant, uncontrollable state of neuron firing. Nevertheless, this condition has a spectrum of findings from merely an annoyance to severely interfering with daily life style or quality of life. Nocturnal RLS causes restless sleep patterns that must be distinguished clinically from other conditions, such as sleep apnea. First described by Ekborn in 1945 (as described, for example, by Pichler et al., Clinical Genetics (2008) 73(4):297-305), it is a neurological disorder characterized by unpleasant sensations and pain, predominantly in the lower extremities at rest, accompanied by an uncontrollable urge for movement for relief. The symptoms are commonly worst at night with accompanying sleep disturbances (Cotter et al., Therapeutics and Clinical Risk Management (2006) 2(4): 465-75). Furthermore, many individuals afflicted with RLS have difficulties with their daily activities, including a mental state of discomfort. The afflicted individuals have therapeutic options, such as dopaminergic, opioid or anticonvulsive compounds, to alleviate their symptoms. These substances have untoward side effects and therefore, many search for other remedies with less adverse reactions, such as alternative natural ingredients. The quest to find a universal, well-tolerated therapy for RLS continues. Although there is no known etiology for RLS, it has been observed that individuals with unhealthy eating habits and poor exercise activities are more likely to develop this condition.

This condition may have a genetic basis, possibly an autosomal dominant trait, although the nature of the genetic defect is unknown, the familial occurrence of this condition is approximately 67%, with women more susceptible than men, which supports the suspected genetic basis. Primary idiopathic RLS usually begins in the early 40's, but has been known in infants, RLS episodes may disappear for months or years. Secondary RLS can usually be traced to specific medical conditions or the use of certain medications, such as anti-nausea drugs, antihistamines, anti-depressive agents, anti-psychotic and anti-seizure drugs. The underlying mechanism for this condition has not yet been established, although theories have been proposed to focus on dopamine and iron systems.

A diagnostic test to identify this condition is missing and therefore the diagnosis is usually made by history, symptoms, and exclusion. The following clinical criteria have been recognized and are commonly used for this diagnosis: (1) the desire to move the limbs, often associated with paresthesias or synesthesias; (2) symptoms that are worse or present only during rest and are partially or temporarily relieved by activity; (3) motor restlessness; and (4) nocturnal worsening of symptoms. RLS affects between 6-15% of the adult populations with an estimated 12 million individuals affected with RLS in the United States. Many researchers believe that this estimate is low due to under diagnoses or misdiagnoses. Many individuals with RLS report socio-economic problems affecting their employment, personal relationships and activities of daily living, which are most likely associated to a lack of restful sleep, chronic exhaustion and inability to focus.

### Case Study Examples

We present a report on two familial males with RLS in which the supplementation of D-ribose produced a benefit in lessening the occurrence and severity of their daily symptoms. Both affected male individuals were from a positive family prevalence, three generations carrying the diagnosis of RLS. These males involved the latter two generations, father and son, ages 71 and 47, respectively. Both men had no underlying medical conditions. Their symptoms of RLS date back for decades and presently have progressed to the moderate to severe discomfort stage, mainly experienced during sedentary periods throughout the day with exacerbation of symptoms during nocturnal sleep.

Both individuals were introduced to D-ribose. To evaluate any potential benefits of D-ribose in RLS, each individual orally consumed D-ribose (five grams per dose) daily at different trial dosing regimens. Each trial dosing regimen lasted for three weeks with a two week interval washout between dosing regimens. The initial regimen involved a single dose of oral D-ribose consumed only at breakfast. The second dosing regimen involved consumption of oral D-ribose at breakfast and lunch. In the last dosing regiment, oral D-ribose was taken with all meals, breakfast, lunch and dinner. A diary pertaining to the documentation and severity of RLS symptoms was compiled.

During the regimen interval, both men reported a general feeling of more energy and less fatigue, most notably after exercise without any significant changes in their symptoms of RLS. However, with the increase in the daily oral dose of D-ribose in the second regimen, their common leg twitching events and the urge to move during the day was reduced for one of the subjects and rarely present in the other. Both still experienced the unpleasant sensations during the night. However, during the final dosing regimen with an increase in the daily oral dose of D-ribose there was total elimination in their diurnal symptoms and the nocturnal symptoms were of a much lesser degree and had a later occurrence.

The two week washout period between stages was designed to observe any return or worsening of their symptoms. Within 48 hours after the cessation of oral D-ribose, both men experienced the return in their clinical RLS symptoms during the day and early evening hours of sleep, which were comparable to their state prior to D-ribose supplementation. Following completion of all dosing regimens of oral D-ribose and with the final wash out period, both males elected to re-start oral dosing of D-ribose at 5 grams/doses three times a day. Both males reported that while D-ribose did not totally eliminate their discomfort, the severity and onset of symptoms affecting their quality of life was substantially improved with D-ribose without any adverse reactions.

Based on the above oral pharmacokinetic information, at a 15 minute half life, the serum D-Ribose levels would drop to near baseline of two mg/dl within an hour, with residual relief lasting several hours. Serum D-ribose levels should be maintained long enough to establish an effect that will persist after the serum D-ribose level is back to baseline levels. The effect in the brain does not persist as long as in the muscles, possibly due to the high levels of energy consumed by the brain throughout the day. Typically, approximately a third of the cardiac output is used to supply energy to the brain.

Further studies are planned to substantiate the findings of the above case study. It is possible that more frequent dosing may further improve results. D-ribose lozenges or gels may make frequent dosing more convenient than dosing at meals. A controlled release product may be most beneficial, especially in relieving the nocturnal symptoms.

### EXAMPLE 2

### Conclusions and Recommendations

The benefit of D-ribose in replenishing ATP levels has been substantiated in numerous studies in healthy individuals undergoing high levels of exercise, and in disease states or conditions of ischemia and hypoxia. Nothing has been known of the effect of D-ribose supplementation on neurological conditions. In the case of RLS, it might be said that a benefit of D-ribose is counterintuitive, since the individuals are not exercising and in fact, exercise relieves the symptoms. The well-known role of D-ribose in replenishing muscle ATP would not seem to be operative.

The most cogent observation from this small study of RLS is that there is very rapid turnover, with washout possibly occurring daily between doses and complete in 48 hours after ceasing D-ribose supplementation. In other studies, such as that of the '942 patent, it has been found that washout requires a minimum of one week and preferably two weeks for the D-Ribose effect to dissipate. Not wishing to be bound by theory, the effect may be due to passage of the D-Ribose into the brain, therein raising ATP levels and improving brain function and that D-Ribose penetrates the BBB only in small amounts, possibly co-transported with glucose through mass action when the serum levels of D-Ribose are high. Therefore, further studies on neurological diseases will employ more frequent administration of D-Ribose or preferably, time-release capsules. D-Ribose has been awarded GRAS status by the United States Food and Drug Administration, is readily administered orally and has no side effects, providing the oral dosage is kept below eight grams at a time, no more often than every two hours. It should be emphasized that this nutritional support is not a cure for the neurological diseases, but is expected to ameliorate symptoms. Neurological disease states expected to benefit from high serum levels of D-Ribose include but are not limited to cerebral palsy, epilepsy, Tourette's syndrome, essential tremors, stroke, aging, senile dementia, Parkinson's disease, depression and schizophrenia.

### EXAMPLE 3

### Cerebral Palsy

Cerebral palsy (CP) findings/symptoms are commonly present at birth, although in mild cases, the diagnosis may not be made for several years following birth. It is thought that CP is caused by damage to the CNS motor/neuronal centers of the developing brain during pregnancy (estimated to be 75% of the cases), during childbirth (5%) or after birth (15%). It may rarely be acquired later in life. CP is a group of permanent disorders of the development of movement and posture, causing activity limitations. The incidence is two to three per thousand births. Babies with very low birth weights are often affected and CP is more common in multiple births.

Seventy to eighty percent of CP patients have spastic palsy, with hypertonia stemming from damage to the motor neurons in the spinal cord and/or to the motor neurons in the cerebrum. The spectrum of motor dysfunction varies from clumsiness to grossly uncoordinated movements.

For patients suffering from cerebral palsy, more frequent pulse 5 gram doses of D-Ribose at least three but up to ten per 24-hour period may be indicated. The exact frequency for dosage may be determined empirically for each patient. Doses should be ingested approximately every five hours with an 8 gram dose at bedtime when the effective total daily dose is determined to be 15 to 20 grams. It is most convenient and also avoids potential hypoglycemia to supplement the oral D-Ribose ingestion with meals. The bedtime dose may be taken with a snack to provide a glucose source.

D-Ribose may be administered in the form of powders, lozenges, capsules or may be incorporated in a liquid drink. Time-release tablets or capsules may maintain the serum levels to the required level when taken fewer times a day. D-ribose can also be administered using an infusible pump as further discussed herein.

### EXAMPLE 4

### Epilepsy

Epilepsy is a chronic neurological disorder characterized by seizures which can be moments of stillness and incomprehension (petit mal) or motor movements of jerking, thrashing and falling (grand mal). These seizures may be totally unprovoked or may be triggered by specific stimuli (sound, light or odors). A number of drugs are on the market to control seizure symptoms and surgical invasive procedures have gained interest into mapping and potentially providing a means for isolated treatment options. However, any role played by nutrition and metabolism has not been totally appreciated.

For mammals (e.g., human patients, horses, dogs) suffering from epilepsy, more frequent pulse 5 gram doses of D-Ribose at least three but up to ten per 24-hour period may be indicated. The exact frequency for dosage may be determined empirically for each mammal. For embodiments in which the mammal is a human patient, the effective total daily dose is determined to be 15 to 20 grams, and doses should be ingested approximately every five hours with an 8 gram dose at bedtime. It is most convenient and also avoids potential hypoglycemia to supplement the oral D-Ribose ingestion with meals. The bedtime dose may be taken with a snack to provide a glucose source.

D-Ribose may be administered in the form of powders, lozenges, capsules or may be incorporated in a liquid drink. Time-release tablets or capsules may maintain the serum levels to the required level when taken fewer times a day. D-ribose can also be administered using an infusible pump as further discussed herein.

The complete disclosures of the patents, patent documents, and publications cited herein are incorporated by reference in their entirety as if each were individually incorporated. Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein as follows.

What is claimed is:

1. A method for treating a human patient having restless legs syndrome, the method comprising administering to the patient in need thereof a therapeutically effective amount of a composition comprising D-ribose.

2. The method of claim 1 wherein administering comprises orally administering.

3. The method of claim 1 wherein administering comprises administering for at least one week.

4. The method of claim 3 wherein administering comprises administering for at least 3 weeks.

5. The method of claim 4 wherein administering comprises chronically administering.

6. The method of claim 2 wherein the therapeutically effective amount of the composition comprises 4 to 50 grams of D-ribose per day.

7. The method of claim 6 wherein the therapeutically effective amount of the composition is administered in 1 to 10 doses per day.

8. The method of claim 7 wherein the therapeutically effective amount of the composition is administered in 1 to 4 doses per day.

9. The method of claim 2 wherein administering the therapeutically effective amount of the composition comprises administering five grams of D-ribose three times a day.

10. The method of claim 9 wherein administering the therapeutically effective amount of the composition consists essentially of administering five grams of D-ribose three times a day.

11. The method of claim 10 wherein administering the therapeutically effective amount of the composition consists of administering five grams of D-ribose three times a day.

12. The method of claim 1 with the proviso that the composition does not comprise folic acid and/or folate.

13. The method of claim 1 with the proviso that the composition does not comprise any of vitamin B6, vitiamin B12, folic acid, or folate.

14. The method of claim 1 with the proviso that the composition does not comprise a B group vitamin.

15. A method for treating a human patient having cerebral palsy, the method comprising administering to the patient in need thereof a therapeutically effective amount of a composition comprising D-ribose.

16. A method for treating a mammal having epilepsy, the method comprising administering to the mammal in need thereof a therapeutically effective amount of a composition comprising D-ribose.

17. A method for treating a mammal suffering from a condition caused by aberrant neuron firing, the method comprising administering to the mammal in need thereof a therapeutically effective amount of a composition comprising D-ribose.

18. The method of claim 17 wherein the mammal is a human patient and administering the therapeutically effective amount of the composition consists of orally administering five grams of D-ribose three times a day.

19. The method of claim 18 wherein orally administering comprises administering for at least three weeks.

20. The method of claim 19 wherein orally administering comprises chronically administering.

21. The method of claim 17 wherein administering to the mammal in need thereof comprises a pulse dosing regimen comprising multiple, repetitive doses for administering D-ribose to a mammal suffering declining health from a condition caused by aberrant neuron firing.

22. The method of claim 21 further comprising escalating the pulse dosing regimen for mammals not responding to the initial pulse dosing regimen.
